(19) United States
(12) Patent Application Publication
(10) Pub. No.: US 2026/0076634 A1
(43) Pub. Date: Mar. 19, 2026

(54) IMAGING OF BRAIN LESIONS

(71) Applicant: Blue Earth Diagnostics Limited, Oxford (GB)

(72) Inventors: Eugene Teoh, Oxford (GB); Robin Holmes, Eynsham Oxfordshire (GB); Sharon Weick, Marlton, NJ (US)

(73) Assignee: Blue Earth Diagnostics Limited, Oxford (GB)

(21) Appl. No.: 19/331,152

(22) Filed: Sep. 17, 2025

Related U.S. Application Data
(60) Provisional application No. 63/695,956, filed on Sep. 18, 2024.

(51) Int. Cl. A61B 6/50; A61B 6/00; A61K 51/04

(52) U.S. Cl. CPC A61B 6/501; A61B 6/483; A61K 51/0402; A61B 2576/026; A61K 2123/00

(57) ABSTRACT

The present disclosure relates to methods of administering [¹⁸F]-FACBC. The present disclosure also relates to use of [¹⁸F]-FACBC in methods for imaging, diagnosing, and monitoring of brain lesions.

### TECHNICAL FIELD

The present invention relates to methods of administering [¹⁸F]-FACBC. The present invention also relates to use of [¹⁸F]-FACBC in methods for imaging and diagnosing brain lesions.

### BACKGROUND

Brain metastases, which may be referred to as secondary brain cancer, occur when cancer cells spread from their original primary tumor site to and/or in the brain. Brain metastases are the most common intracranial tumor in adults, occurring in up to 40% of patients with cancer, with approximately 200,000 patients affected each year in the USA. Following localized treatment of brain metastases (increasingly, stereotactic radiosurgery [SRS] alongside neurosurgical resection), close follow-up with serial magnetic resonance imaging (MRI) of the brain is performed to evaluate for recurrent disease. Conventional MRI is currently recommended as the main imaging test (NCCN, 2019) following localized treatment, as it is widely available and offers high spatial resolution, with presence of recurrent disease suggested by increased contrast enhancement (CE) depicting anatomical/structural information. However, conventional MRI (CE-T1 and fluid attenuated inversion recovery [FLAIR]/T2-weighted sequences) has limited specificity due to the incidence of treatment-related changes, including radiation necrosis. These treatment-related changes have similar appearance to true recurrence of disease on conventional MRI, including CE, origin near the primary tumor site, vasogenic edema, growth over time, and mass effect. The same issue of recurrence compared to treatment related changes is also experienced in primary brain tumors, where differentiating between true progression and pseudoprogression presents the same diagnostic challenges.

No specific feature or combination of features on conventional MRI has been established to differentiate between disease recurrence and treatment-related changes including radiation necrosis and pseudo-progression.

Given this area of great diagnostic unmet need, accurate imaging to differentiate disease recurrence from treatment-related changes is valuable for several reasons:
Identifying treatment-related changes is important to avoid unneeded treatment (e.g., surgery) and erroneously premature termination of potentially effective treatment.
Accurate scans can inform the management decision of cessation of non-effective treatments, to minimize morbidity from treatment side effects and reduce the economic burden.
Timely diagnosis of true recurrence can facilitate prompt stratification of patients to further therapies, which may maximize therapeutic benefit and clinical outcome.
Given the high morbidity and mortality of patients with brain metastases and, therefore, the need for high quality clinical research, such imaging may be pivotal in determining suitability for clinical trial entry, and accurate characterization of investigational therapeutic efficacy.
Aiding the physician to risk-stratify continuation of a therapeutic regimen (where treatment-related changes can be confidently diagnosed). This may be of particular value in the context of a treatment with a significant side effect profile.

[¹⁸F]-fluoro-2-deoxy-glucose (FDG) is a PET imaging agent for the detection and localisation of many forms of cancer. However, FDG-PET has been found to have less sensitivity and/or specificity for assessment of some types of cancer, for example, brain tumors.

Metastasis involves a complex series of steps in which cancer cells leave the primary tumor site and migrate to other parts of the body via the bloodstream or the lymphatic system. The new occurrences of tumor thus generated are referred to as a metastatic lesion or metastasis. Metastatic lesions are very common in the late stages of cancer and are a major cause of death from solid lesions. There are known difficulties associated with diagnosing and monitoring metastatic cancer.

Thus, there is a need for a method of imaging which allows for reproducible, reliable imaging for detection and monitoring of metastatic cancer in the brain.

Thus, there is a need for a method of reproducible, reliable imaging of metastatic cancer in the brain to permit diagnosis of metastatic cancer in the brain.

Thus, there is a need for a method of reproducible, reliable imaging of metastatic cancer in the brain to permit and/or facilitate distinction between metastatic cancer, particularly recurrent metastatic cancer, in the brain and treatment-related changes in the brain, such as radiation necrosis and pseudo-progression.

### SUMMARY

In one aspect of the disclosure, there is provided a method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) visually analysing the one or more PET scan images by comparing [¹⁸F]-FACBC uptake in a potential lesion of the head region of the subject against [¹⁸F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [¹⁸F]-FACBC uptake in a potential lesion of the head region of the subject against [¹⁸F]-FACBC uptake of the parotid gland. If criterion of (i) is not met, comparing [¹⁸F]-FACBC uptake in a potential lesion of the head region of the subject against [¹⁸F]-FACBC uptake of the pituitary in the head region, and
(d) determining whether the potential lesion is a brain lesion based on the analysis of step (c).

In some embodiments, step (c) of the method uses either the parotid gland or pituitary as reference organs for visual image interpretation of FACBC images of lesions. Specific instruction is given as to whether to use the parotid gland or pituitary as the reference organ, based on a minimum of predetermined size of craniocaudally visualised parotid gland on the scan field of view. If this criterion is met, the parotid gland is used as the reference organ. If it is not met, the pituitary should be used as the reference organ.

In some embodiments, the brain lesion is a primary brain lesion. In some embodiments, the brain lesion is a metastatic brain lesion.

In some embodiments, the predetermined size of craniocaudally visualised parotid gland on the scan field of view is about 0.7-0.9 cm, for example 0.8 cm. In some embodiments, the predetermined size of craniocaudally visualised parotid gland on the scan field of view is about 0.9-1.1 cm, for example 1.0 cm. In some embodiments, the predetermined size of craniocaudally visualised parotid gland on the scan field of view is about 1.1-1.3 cm, for example 1.2 cm. In some embodiments, the predetermined size of craniocaudally visualised parotid gland on the scan field of view is about 1.3-1.5 cm, for example 1.4 cm. In some embodiments, the predetermined size of craniocaudally visualised parotid gland on the scan field of view is about 1.5-1.7 cm, for example 1.6 cm. In some embodiments, the predetermined size of craniocaudally visualised parotid gland on the scan field of view is about 1.7-1.9 cm, for example 1.8 cm. In some embodiments, the predetermined size of craniocaudally visualised parotid gland on the scan field of view is about 1.9-2.1 cm, for example 2.0 cm. In some embodiments, the predetermined size of craniocaudally visualised parotid gland on the scan field of view is about 2.1-2.3 cm, for example 2.2 cm. In some embodiments, the predetermined size of craniocaudally visualised parotid gland on the scan field of view is about 2.3-2.5 cm, for example 2.4 cm. In some embodiments, the predetermined size of craniocaudally visualised parotid gland on the scan field of view is about 2.5-2.7 cm, for example 2.6 cm. In some embodiments, the predetermined size of craniocaudally visualised parotid gland on the scan field of view is about 2.7-2.9 cm, for example 2.8 cm. In some embodiments, the predetermined size of craniocaudally visualised parotid gland on the scan field of view is about 2.9-3.1 cm, for example 3.0 cm.

In some embodiments, the method instructs the reader to interpret the lesion as recurrence (positive) if the lesion FACBC uptake is equal to or higher than the reference organ, and treatment-related change (negative) if the lesion FACBC uptake is less than the reference organ.

In some embodiments, comparison of lesion uptake is specifically intended to be the area of highest FACBC uptake within the lesion, compared to the visual average of the reference organ.

In some embodiments, instruction is given to making this comparison and is done based on adjustment of the image contrast (“windowing”) to a specific visual rendering of the reference organ. Once this is achieved, the visual average of the reference organ is considered to be at a fixed point of the image scale (“lookup table”/“colour scale”). In some embodiment, the fixed point is about ¾ point of the image scale. In some embodiment, the fixed point is about ⅘ point of the image scale. In some embodiment, the fixed point is about ⅚ point of the image scale. If the lesion uptake (i.e. the area of highest FACBC uptake within the lesion) is visually equal to or higher than this point, then the lesion meets the criterion for positive.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

In another aspect of the disclosure, there is provided a semi-quantitative method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) analysing the one or more PET scan images by comparing a standardized uptake value (SUV) of [¹⁸F]-FACBC uptake in a potential lesion against a reference standardized uptake value (SUV)_{threshold} of the pituitary gland; and
(d) determining whether the potential lesion is a brain lesion based on the analysis of step (c).

In some embodiments of this aspect of invention, one volume of interest (VOI) each is drawn around the lesion, and a separate VOI is drawn around the reference organ using an image workstation. The level of FACBC uptake within these structures are characterised by the hottest (highest level of FACBC uptake) cubic centimetre within the VOL. In some embodiments, the level of FACBC uptake within these structures are characterised by the hottest voxel within the VOI, also known as SUVmax. In some embodiments, the level of FACBC uptake within these structures are characterised by the average of FACBC uptake within the VOI, also known as SUVmean. In some embodiments, the level of uptake is characterised numerically by the standardized uptake value, also known as the peak standardized uptake value (SUVpeak). By the new method, the lesion uptake is compared to pituitary uptake, generating an SUV ratio (SUVR_{lesion:pituitary}). Where the ratio is ≥1, the lesion is classified as positive. Where the ratio is <1, the lesion is classified as negative.

In some embodiments, the brain lesion is a primary brain lesion. In some embodiments, the brain lesion is a metastatic brain lesion.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

In another aspect of the invention, there is provided a method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) visually analysing the one or more PET scan images by comparing [¹⁸F]-FACBC uptake in a potential lesion of the head region of the subject against [¹⁸F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [¹⁸F]-FACBC uptake in a potential lesion of the head region of the subject against [¹⁸F]-FACBC uptake of the parotid gland. If criterion of (i) is not met, comparing [¹⁸F]-FACBC uptake in a potential lesion of the head region of the subject against [¹⁸F]-FACBC uptake of the pituitary in the head region;
(d) if in step (c) the [¹⁸F]-FACBC uptake in a potential lesion of the head region of the subject is visually close to [¹⁸F]-FACBC uptake in parotid glands or pituitary of the head region, comparing a standardized uptake value (SUV) of [¹⁸F]-FACBC uptake in a potential lesion against a reference threshold standardized uptake value (SUV)_{threshold} of the pituitary, and
(e) determining whether the potential lesion is a metastatic brain lesion based on the analysis of step (c) and (d).

In this aspect of the invention, the method incorporates use of the two aforementioned methods, where a reader may elect to use the parotid as reference organ for visual image interpretation, and where there is insufficient parotid gland in the scan field of view, use the pituitary as reference organ for visual image interpretation. If the [¹⁸F]-FACBC uptake in a potential lesion of the head region of the subject is visually close to [¹⁸F]-FACBC uptake in the chosen reference organ, semi-quantitative SUVR method will be used with the pituitary gland as the reference organ.

In some embodiments, the brain lesion is a primary brain lesion. In some embodiments, the brain lesion is a metastatic brain lesion.

In some embodiments, the predetermined size of craniocaudally visualised parotid gland on the scan field of view is about 0.7-0.9 cm, for example 0.8 cm. In some embodiments, the predetermined size of craniocaudally visualised parotid gland on the scan field of view is about 0.9-1.1 cm, for example 1.0 cm. In some embodiments, the predetermined size of craniocaudally visualised parotid gland on the scan field of view is about 1.1-1.3 cm, for example 1.2 cm. In some embodiments, the predetermined size of craniocaudally visualised parotid gland on the scan field of view is about 1.3-1.5 cm, for example 1.4 cm. In some embodiments, the predetermined size of craniocaudally visualised parotid gland on the scan field of view is about 1.5-1.7 cm, for example 1.6 cm. In some embodiments, the predetermined size of craniocaudally visualised parotid gland on the scan field of view is about 1.7-1.9 cm, for example 1.8 cm. In some embodiments, the predetermined size of craniocaudally visualised parotid gland on the scan field of view is about 1.9-2.1 cm, for example 2.0 cm. In some embodiments, the predetermined size of craniocaudally visualised parotid gland on the scan field of view is about 2.1-2.3 cm, for example 2.2 cm. In some embodiments, the predetermined size of craniocaudally visualised parotid gland on the scan field of view is about 2.3-2.5 cm, for example 2.4 cm. In some embodiments, the predetermined size of craniocaudally visualised parotid gland on the scan field of view is about 2.5-2.7 cm, for example 2.6 cm. In some embodiments, the predetermined size of craniocaudally visualised parotid gland on the scan field of view is about 2.7-2.9 cm, for example 2.8 cm. In some embodiments, the predetermined size of craniocaudally visualised parotid gland on the scan field of view is about 2.9-3.1 cm, for example 3.0 cm.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

In another aspect of the invention, there is provided a method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) visually analysing the one or more PET scan images by comparing [¹⁸F]-FACBC uptake in a potential lesion of the head region of the subject against [¹⁸F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [¹⁸F]-FACBC uptake in a potential lesion of the head region of the subject against [¹⁸F]-FACBC uptake of the parotid gland;
(d) if in step (c) the criterion of (i) is not met, comparing a standardized uptake value (SUV) of [¹⁸F]-FACBC uptake in a potential lesion against a reference threshold standardized uptake value (SUV)_{threshold} of the pituitary, and
(e) determining whether the potential lesion is a metastatic brain lesion based on the analysis of step (c) and (d).

In this aspect of the invention, a reader may elect to use the parotid as reference organ for visual image interpretation, and where there is insufficient parotid gland in the scan field of view, the semi-quantitative SUVR method will be used with the pituitary gland as the reference organ.

In some embodiments, the brain lesion is a primary brain lesion. In some embodiments, the brain lesion is a metastatic brain lesion.

In some embodiments, the predetermined size of craniocaudally visualised parotid gland on the scan field of view is about 0.7-0.9 cm, for example 0.8 cm. In some embodiments, the predetermined size of craniocaudally visualised parotid gland on the scan field of view is about 0.9-1.1 cm, for example 1.0 cm. In some embodiments, the predetermined size of craniocaudally visualised parotid gland on the scan field of view is about 1.1-1.3 cm, for example 1.2 cm. In some embodiments, the predetermined size of craniocaudally visualised parotid gland on the scan field of view is about 1.3-1.5 cm, for example 1.4 cm. In some embodiments, the predetermined size of craniocaudally visualised parotid gland on the scan field of view is about 1.5-1.7 cm, for example 1.6 cm. In some embodiments, the predetermined size of craniocaudally visualised parotid gland on the scan field of view is about 1.7-1.9 cm, for example 1.8 cm. In some embodiments, the predetermined size of craniocaudally visualised parotid gland on the scan field of view is about 1.9-2.1 cm, for example 2.0 cm. In some embodiments, the predetermined size of craniocaudally visualised parotid gland on the scan field of view is about 2.1-2.3 cm, for example 2.2 cm. In some embodiments, the predetermined size of craniocaudally visualised parotid gland on the scan field of view is about 2.3-2.5 cm, for example 2.4 cm. In some embodiments, the predetermined size of craniocaudally visualised parotid gland on the scan field of view is about 2.5-2.7 cm, for example 2.6 cm. In some embodiments, the predetermined size of craniocaudally visualised parotid gland on the scan field of view is about 2.7-2.9 cm, for example 2.8 cm. In some embodiments, the predetermined size of craniocaudally visualised parotid gland on the scan field of view is about 2.9-3.1 cm, for example 3.0 cm.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

### DETAILED DESCRIPTION OF THE INVENTION

The features of the invention relating, where appropriate to each and all aspects of the invention, will now be described in more detail as follows.

### BRIEF DESCRIPTION OF DRAWINGS

FIG. 1. Example PET transaxial slices at the bottom of the PET camera field of view. The top row shows usable slices. The bottom row shows noisy slices at the edge of field of view that cannot be used.

FIG. 2. Illustration of suitable windowing for reference organ/lesion comparison.

FIG. 3. Illustration of different window levels for the parotid glands. The left-hand images show an upper window that is too low and the right images show an upper window that is too high.

FIG. 4. Illustration of different window levels for the pituitary. The left-hand images show an upper window that is too low and the right images show an upper window that is too high.

FIG. 5. Illustration of suitable windowing for reference organ/lesion comparison. Additionally, the box in dotted lines shows the zone where lesion may be classified as visually close to the reference organ.

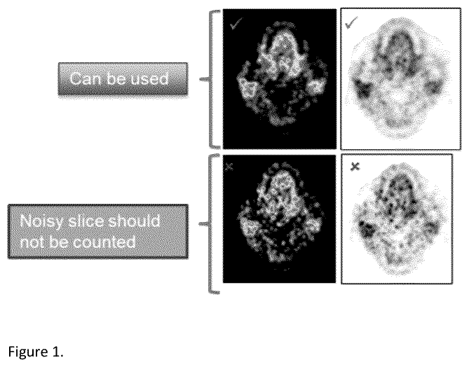

### DEFINITIONS

In this specification, the following words and expressions, if and when used, shall have the meanings ascribed below:

singular forms “a,” “an,” and “the” include plural reference unless the context dictates otherwise;

“approximately” and “about” refer to being nearly the same as a referenced number or value (e.g. substantially the same as without affecting the material aspect(s) of the invention). The terms “approximately” and “about” generally should be understood to encompass ±5% of a specified amount or value;

“comprising” or any cognate word specifies the presence of stated features, steps, or integers or components, but does not preclude the presence or addition of one or more other features, steps, integers, components or groups thereof. The expressions “consists of” or “consists essentially of” or cognates may be embraced within “comprises” or any cognate word. The expression “consists essentially of” permits inclusion of substances not materially affecting the characteristics of the composition to which it applies. The expression “consists of” or cognates means only the stated features, steps, integers components or groups thereof are present to which the expression refers;

“detectable effective amount” in respect of the amount of [¹⁸F]-FACBC administered to a subject means an amount of [¹⁸F]-FACBC, preferably administered by intravenous administration, that is effective to provide, and provides, uptake by a potential lesion to allow acquisition of a PET image of the potential lesion. An exemplary detectable amount of [¹⁸F]-FACBC is 185 MBq (mega bequerels)±20%;

“standardised uptake value (SUV)” in respect of uptake of [¹⁸F]-FACBC by a potential lesion or other region (e.g. venous sinus, pituitary and/or parotid glands) of the head represents the value obtained from the ratio of the decay corrected amount of radioactivity uptake by the potential lesion (or uptake by the other region of interest) in the head, typically measured in Bq/ml, to the total amount of radioactivity administered to a subject, typically measured in Bq, multiplied by the body weight of the subject, typically measured in grams;

“maximum standardised uptake value (SUV).” in respect of uptake of [¹⁸F]-FACBC by a potential lesion or other region (e.g. venous sinus, pituitary, parotid glands) of the head is defined as the standardised uptake value (SUV) measured at a point in the potential lesion (or other region) of the head, respectively, where maximum uptake of [¹⁸F]-FACBC occurs. The point where maximum uptake of [¹⁸F]-FACBC occurs in the potential lesion (or other region) of the head represents the highest intensity point (“hottest spot”) in the PET image for the potential lesion or other region. Suitably, (SUV)ₘₐₓ corresponds to (SUV) at the hottest spot in the PET image;

“peak standardised uptake value (SUV)ₚₑₐₖ” in respect of uptake of [¹⁸F]-FACBC by a potential lesion or other region (e.g. venous sinus, pituitary, parotid glands) of the head is defined as the mean standardised uptake value (SUV) in a 1 cm³ spherical volume which sphere is centred on the point where maximum uptake of [¹⁸F]-FACBC occurs in the potential lesion (or other region), i.e. centred on the point in the potential lesion (or other region) where (SUV)ₘₐₓ occurs and is measured;

“mean standardized uptake value (SUV).” represents the average standardized uptake value of [¹⁸F]-FACBC within a defined volume of a potential lesion or other region (e.g. venous sinus) of the head;

“reference threshold maximum standardized uptake value (SUV)_{threshold-max}” represents the lower, including lowest, limit maximum standardized uptake value of a potential lesion such that a potential lesion in the head having a measured maximum standardized uptake value (SUV)ₘₐₓ greater than or equal to reference threshold maximum standardized uptake value (SUV)_{threshold-max} is identifiable as a brain lesion;

“reference threshold peak standardized uptake value (SUV)_{threshold-peak}” represents the lower, including lowest, limit peak standardized uptake value of a potential lesion such that a potential lesion in the head having a measured peak standardized uptake value (SUV)ₚₑₐₖ greater than or equal to reference threshold peak standardized uptake value (SUV)_{threshold-peak} is identifiable as a brain lesion;

“standardised uptake value (SUV)”, “maximum standardised uptake value (SUV)ₘₐₓ”, “peak standardised uptake value (SUV)ₚₑₐₖ”, and “mean standardized uptake value (SUV)ₘₑₐₙ” of a radiotracer represent known parameters to those skilled in the art and can be measured quantitatively using standard functionality of commercially available PET imaging workstations, e.g. by placing a region/volume-of-interest tool on the potential lesion (or other region of the head), which then calculates the (SUV), (SUV)ₘₐₓ, (SUV)ₚₑₐₖ”, and (SUV)ₘₑₐₙ values;

“commercially available standard PET imaging workstations” includes MIM® software, HERMIA (Hermes Medical Solutions), GE Healthcare Advantage Workstation and Siemens Healthcare;

Further, it is understood that any upper and lower quantity, range and ratio limits set forth herein may be independently combined. Accordingly, any upper and lower quantity, range and ratio limits set forth herein associated with a particular technical feature of the present invention may be independently combined with any upper and lower quantity, range and ratio limits set forth herein associated with one or more other particular technical feature(s) of the present invention. Furthermore, any particular technical feature of the present invention, and all preferred variants thereof, may be independently combined with any other particular technical feature(s), and all preferred variants thereof, irrespective of whether such features are presented as preferred or not.

Also, it will be understood that the preferred features of each aspect of the present invention are regarded as preferred features of each and every aspect of the present invention.

Fluciclovine (¹⁸F) injection, also known as [¹⁸F]-FACBC, FACBC, or anti-1-amino-3-¹⁸F-fluorocyclobutane-1-carboxylic acid, is a synthetic amino acid imaging agent which is taken up specifically by amino acid transporters and is used for positron emission tomography (PET). PET is uniquely suited to evaluate metabolic activity in human tissue for diagnostic imaging purposes. Use of ¹⁸F-fluciclovine in the imaging of brain metastases is of considerable clinical relevance. ¹⁸F-Fluciclovine has utility in the evaluation of primary and metastatic cancers in the brain due at least in part to its low normal brain background uptake and increased uptake by brain tumors.

The present inventors have established a protocol which allows for the imaging of brain lesions, e.g., providing greater reliability and reproducibility, allowing the data from PET scan images to be analysed and compared in order to accurately diagnose or monitor brain lesions.

Aspects of the present invention relate to methods of diagnosing brain lesions, e.g., analysing potential lesions to determine whether they are brain lesions, in a subject by administering anti-1-amino-3-¹⁸F-fluorocyclobutane-1-carboxylic acid ([¹⁸F]-FACBC) for improved PET imaging and more reliable diagnosis of cancer and the metastasis or recurrence thereof. For example, the present invention relates to methods of administering [¹⁸F]-FACBC for improved PET imaging and more reliable diagnosis of brain lesions and the metastasis or recurrence thereof. The present invention further relates to methods of diagnosing brain lesions and the metastasis or recurrence thereof, using the PET imaging agent [¹⁸F]-FACBC. Still further, the present invention relates to methods of identifying and diagnosing metastatic brain lesion(s), and the recurrence thereof, using the PET imaging agent [¹⁸F]-FACBC, and permitting distinction between the metastatic brain lesion(s) and treatment-related changes in the brain, such as radiation necrosis and pseudo-progression.

In one aspect of the disclosure, there is provided a method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) visually analysing the one or more PET scan images by comparing [¹⁸F]-FACBC uptake in a potential lesion of the head region of the subject against [¹⁸F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [¹⁸F]-FACBC uptake in a potential lesion of the head region of the subject against [¹⁸F]-FACBC uptake of the parotid gland. If criterion of (i) is not met, comparing [¹⁸F]-FACBC uptake in a potential lesion of the head region of the subject against [¹⁸F]-FACBC uptake of the pituitary in the head region, and
(d) determining whether the potential lesion is a brain lesion based on the analysis of step (c).

In some embodiments, step (c) of the method uses either the parotid gland or pituitary as reference organs for visual image interpretation of FACBC images of lesions. Specific instruction is given as to whether to use the parotid gland or pituitary as the reference organ, based on a minimum of predetermined size of craniocaudally visualised parotid gland on the scan field of view. If this criterion is met, the parotid gland is used as the reference organ. If it is not met, the pituitary should be used as the reference organ.

In some embodiments, the brain lesion is a primary brain lesion. In some embodiments, the brain lesion is a metastatic brain lesion.

In some embodiments, the predetermined size of craniocaudally visualised parotid gland on the scan field of view is about 0.7-0.9 cm, for example 0.8 cm. In some embodiments, the predetermined size of craniocaudally visualised parotid gland on the scan field of view is about 0.9-1.1 cm, for example 1.0 cm. In some embodiments, the predetermined size of craniocaudally visualised parotid gland on the scan field of view is about 1.1-1.3 cm, for example 1.2 cm. In some embodiments, the predetermined size of craniocaudally visualised parotid gland on the scan field of view is about 1.3-1.5 cm, for example 1.4 cm. In some embodiments, the predetermined size of craniocaudally visualised parotid gland on the scan field of view is about 1.5-1.7 cm, for example 1.6 cm. In some embodiments, the predetermined size of craniocaudally visualised parotid gland on the scan field of view is about 1.7-1.9 cm, for example 1.8 cm. In some embodiments, the predetermined size of craniocaudally visualised parotid gland on the scan field of view is about 1.9-2.1 cm, for example 2.0 cm. In some embodiments, the predetermined size of craniocaudally visualised parotid gland on the scan field of view is about 2.1-2.3 cm, for example 2.2 cm. In some embodiments, the predetermined size of craniocaudally visualised parotid gland on the scan field of view is about 2.3-2.5 cm, for example 2.4 cm. In some embodiments, the predetermined size of craniocaudally visualised parotid gland on the scan field of view is about 2.5-2.7 cm, for example 2.6 cm. In some embodiments, the predetermined size of craniocaudally visualised parotid gland on the scan field of view is about 2.7-2.9 cm, for example 2.8 cm. In some embodiments, the predetermined size of craniocaudally visualised parotid gland on the scan field of view is about 2.9-3.1 cm, for example 3.0 cm.

In some embodiments, the method instructs the reader to interpret the lesion as recurrence (positive) if the lesion FACBC uptake is equal to or higher than the reference organ, and treatment-related change (negative) if the lesion FACBC uptake is less than the reference organ.

In some embodiments, comparison of lesion uptake is specifically intended to be the area of highest FACBC uptake within the lesion, compared to the visual average of the reference organ.

In some embodiments, instruction is given to making this comparison and is done based on adjustment of the image contrast (“windowing”) to a specific visual rendering of the reference organ. Once this is achieved, the visual average of the reference organ is considered to be at a fixed point of the image scale (“lookup table”/“colour scale”). In some embodiment, the fixed point is about ¾ point of the image scale. In some embodiment, the fixed point is about ⅘ point of the image scale. In some embodiment, the fixed point is about ⅚ point of the image scale. If the lesion uptake (i.e. the area of highest FACBC uptake within the lesion) is visually equal to or higher than this point, then the lesion meets the criterion for positive.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

The benefits of this approach are that visual assessment of images are the primary approach used by imaging physicians, and in line with accustomed practice.

In another aspect of the disclosure, there is provided a method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) analysing the one or more PET scan images by comparing a standardized uptake value (SUV) of [¹⁸F]-FACBC uptake in a potential lesion against a reference standardized uptake value (SUV)_{threshold} of the pituitary gland; and
(d) determining whether the potential lesion is a brain lesion based on the analysis of step (c).

In some embodiments of this aspect of invention, one volume of interest (VOI) each is drawn around the lesion, and a separate VOI is drawn around the reference organ using an image workstation. The level of FACBC uptake within these structures are characterised by the hottest (highest level of FACBC uptake) cubic centimetre within the VOL. In some embodiments, the level of FACBC uptake within these structures are characterised by the hottest voxel within the VOI, also known as SUVmax. In some embodiments, the level of FACBC uptake within these structures are characterised by the average of FACBC uptake within the VOI, also known as SUVmean. In some embodiments, the level of uptake is characterised numerically by the standardized uptake value, also known as the peak standardized uptake value (SUVpeak). By the new method, the lesion uptake is compared to pituitary uptake, generating an SUV ratio (SUVR_{lesion:pituitary}). Where the ratio is ≥1, the lesion is classified as positive. Where the ratio is <1, the lesion is classified as negative.

In some embodiments, the brain lesion is a primary brain lesion. In some embodiments, the brain lesion is a metastatic brain lesion.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

In another aspect of the invention, there is provided a method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) visually analysing the one or more PET scan images by comparing [¹⁸F]-FACBC uptake in a potential lesion of the head region of the subject against [¹⁸F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [¹⁸F]-FACBC uptake in a potential lesion of the head region of the subject against [¹⁸F]-FACBC uptake of the parotid gland. If criterion of (i) is not met, comparing [¹⁸F]-FACBC uptake in a potential lesion of the head region of the subject against [¹⁸F]-FACBC uptake of the pituitary in the head region;
(d) if in step (c) the [¹⁸F]-FACBC uptake in a potential lesion of the head region of the subject is visually close to [¹⁸F]-FACBC uptake in parotid glands or pituitary of the head region, comparing a standardized uptake value (SUV) of [¹⁸F]-FACBC uptake in a potential lesion against a reference threshold standardized uptake value (SUV)_{threshold} of the pituitary, and
(e) determining whether the potential lesion is a metastatic brain lesion based on the analysis of step (c) and (d).

In this aspect of the invention, the method incorporates use of the two aforementioned methods, where a reader may elect to use the parotid as reference organ for visual image interpretation, and where there is insufficient parotid gland in the scan field of view, use the pituitary as reference organ for visual image interpretation. If the [¹⁸F]-FACBC uptake in a potential lesion of the head region of the subject is visually close to [¹⁸F]-FACBC uptake in the chosen reference organ, semi-quantitative SUVR method will be used with the pituitary gland as the reference organ.

In some embodiments, the brain lesion is a primary brain lesion. In some embodiments, the brain lesion is a metastatic brain lesion.

In some embodiments, the predetermined size of craniocaudally visualised parotid gland on the scan field of view is about 0.7-0.9 cm, for example 0.8 cm. In some embodiments, the predetermined size of craniocaudally visualised parotid gland on the scan field of view is about 0.9-1.1 cm, for example 1.0 cm. In some embodiments, the predetermined size of craniocaudally visualised parotid gland on the scan field of view is about 1.1-1.3 cm, for example 1.2 cm. In some embodiments, the predetermined size of craniocaudally visualised parotid gland on the scan field of view is about 1.3-1.5 cm, for example 1.4 cm. In some embodiments, the predetermined size of craniocaudally visualised parotid gland on the scan field of view is about 1.5-1.7 cm, for example 1.6 cm. In some embodiments, the predetermined size of craniocaudally visualised parotid gland on the scan field of view is about 1.7-1.9 cm, for example 1.8 cm. In some embodiments, the predetermined size of craniocaudally visualised parotid gland on the scan field of view is about 1.9-2.1 cm, for example 2.0 cm. In some embodiments, the predetermined size of craniocaudally visualised parotid gland on the scan field of view is about 2.1-2.3 cm, for example 2.2 cm. In some embodiments, the predetermined size of craniocaudally visualised parotid gland on the scan field of view is about 2.3-2.5 cm, for example 2.4 cm. In some embodiments, the predetermined size of craniocaudally visualised parotid gland on the scan field of view is about 2.5-2.7 cm, for example 2.6 cm. In some embodiments, the predetermined size of craniocaudally visualised parotid gland on the scan field of view is about 2.7-2.9 cm, for example 2.8 cm. In some embodiments, the predetermined size of craniocaudally visualised parotid gland on the scan field of view is about 2.9-3.1 cm, for example 3.0 cm.

In some embodiments of this aspect of invention, in step (d) of this method, one volume of interest (VOI) each is drawn around the lesion, and a separate VOI is drawn around the reference organ using an image workstation. The level of FACBC uptake within these structures are characterised by the hottest (highest level of FACBC uptake) cubic centimetre within the VOL. In some embodiments, the level of FACBC uptake within these structures are characterised by the hottest voxel within the VOI, also known as SUVmax. In some embodiments, the level of FACBC uptake within these structures are characterised by the average of FACBC uptake within the VOI, also known as SUVmean. In some embodiments, the level of uptake is characterised numerically by the standardized uptake value, also known as the peak standardized uptake value (SUVpeak). By the new method, the lesion uptake is compared to pituitary uptake, generating an SUV ratio (SUVR_{lesion:pituitary}). Where the ratio is ≥1, the lesion is classified as positive. Where the ratio is <1, the lesion is classified as negative.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

In another aspect of the invention, there is provided a method of diagnosing brain lesion(s) in a subject comprising the steps of
(a) administering a detectable amount of [¹⁸F]-FACBC to a subject;
(b) acquiring one or more positron emission tomography (PET) scan images of a head region of the subject;
(c) visually analysing the one or more PET scan images by comparing [¹⁸F]-FACBC uptake in a potential lesion of the head region of the subject against [¹⁸F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [¹⁸F]-FACBC uptake in a potential lesion of the head region of the subject against [¹⁸F]-FACBC uptake of the parotid gland;
(d) if in step (c) the criterion of (i) is not met, comparing a standardized uptake value (SUV) of [¹⁸F]-FACBC uptake in a potential lesion against a reference threshold standardized uptake value (SUV)_{threshold} of the pituitary, and
(e) determining whether the potential lesion is a metastatic brain lesion based on the analysis of step (c) and (d).

In this aspect of the invention, a reader may elect to use the parotid as reference organ for visual image interpretation, and where there is insufficient parotid gland in the scan field of view, the semi-quantitative SUVR method will be used with the pituitary gland as the reference organ.

In some embodiments, the brain lesion is a primary brain lesion. In some embodiments, the brain lesion is a metastatic brain lesion.

In some embodiments, the predetermined size of craniocaudally visualised parotid gland on the scan field of view is about 0.7-0.9 cm, for example 0.8 cm. In some embodiments, the predetermined size of craniocaudally visualised parotid gland on the scan field of view is about 0.9-1.1 cm, for example 1.0 cm. In some embodiments, the predetermined size of craniocaudally visualised parotid gland on the scan field of view is about 1.1-1.3 cm, for example 1.2 cm. In some embodiments, the predetermined size of craniocaudally visualised parotid gland on the scan field of view is about 1.3-1.5 cm, for example 1.4 cm. In some embodiments, the predetermined size of craniocaudally visualised parotid gland on the scan field of view is about 1.5-1.7 cm, for example 1.6 cm. In some embodiments, the predetermined size of craniocaudally visualised parotid gland on the scan field of view is about 1.7-1.9 cm, for example 1.8 cm. In some embodiments, the predetermined size of craniocaudally visualised parotid gland on the scan field of view is about 1.9-2.1 cm, for example 2.0 cm. In some embodiments, the predetermined size of craniocaudally visualised parotid gland on the scan field of view is about 2.1-2.3 cm, for example 2.2 cm. In some embodiments, the predetermined size of craniocaudally visualised parotid gland on the scan field of view is about 2.3-2.5 cm, for example 2.4 cm. In some embodiments, the predetermined size of craniocaudally visualised parotid gland on the scan field of view is about 2.5-2.7 cm, for example 2.6 cm. In some embodiments, the predetermined size of craniocaudally visualised parotid gland on the scan field of view is about 2.7-2.9 cm, for example 2.8 cm. In some embodiments, the predetermined size of craniocaudally visualised parotid gland on the scan field of view is about 2.9-3.1 cm, for example 3.0 cm.

In some embodiments, FACBC brain images of the methods may be obtained about 5-15 minutes post-injection of FACBC. In some embodiments, FACBC brain images of the methods may be obtained about 10-20 minutes post-injection of FACBC. In some embodiments, FACBC brain images of the methods may be obtained about 15-25 minutes post-injection of FACBC. In some embodiments, FACBC brain images of the methods may be obtained about 20-30 minutes post-injection of FACBC. In some embodiments, FACBC brain images of the methods may be obtained about 15-30 minutes post-injection of FACBC.

Alternatively, the reader may use the visual approach, and confirm their visual interpretation using the semi-quantitative SUVR approach to increase the confidence of their interpretation.

Alternatively, the reader may use the visual approach, and deems the image to be a borderline assessment or the lesion FACBC uptake is visually close to the reference organ, and proceeds to use the SUVR approach to aid their interpretation.

In particular, although not exclusively, the present invention may include making a comparison of a standardized uptake value (SUV) of the [¹⁸F]-FACBC uptake in a potential lesion against a reference threshold standardized uptake value (SUV)_{threshold}. Typically, this comparison may allow for a more accurate diagnosis of the lesion than using MRI and/or CT alone, e.g. diagnosing brain lesions and/or the metastasis or recurrence thereof.

The method of the invention may be further used to diagnose a brain lesion, such as a metastatic brain lesion or a recurrence of primary brain tumors, in a subject. For example, the brain lesion may comprise a cancer lesion, such as a metastatic cancer lesion.

The method of the invention may be further used to distinguish between metastatic cancer, particularly recurrent metastatic cancer, in the brain and treatment-related changes in the brain, such as radiation necrosis and pseudo-progression.

Accordingly, the present invention, as defined and identified herein, may be used for diagnosing a brain lesion, such as a metastatic cancer lesion in the brain, of a subject. The present invention, as defined and identified herein, provides a method of imaging which typically allows for reproducible, reliable imaging for detection and monitoring of metastatic cancer in the brain of a subject. The present invention, as defined herein, typically provides a reproducible and reliable method to distinguish between metastatic cancer in the brain and disease recurrence and an artefact(s) of treatment-related changes in the brain, such as radiation necrosis and pseudo-progression.

Suitably, references to in-vivo diagnosis method(s) of the present invention are also to be interpreted as references to compounds, diagnostic compositions and medicaments of the invention for use in said method(s) of diagnosis.

The methods herein may include, for example, instructions for the subject to consume no food or calorie-containing drink during a period of time prior to the administration of the [¹⁸F]-FACBC, the subject having followed the instructions. The period of time may be, e.g., at least 4 hours (e.g., 4 continuous hours) or at least 6 hours (e.g., 6 continuous hours) prior to the administration of the [¹⁸F]-FACBC.

For example, for a period of time prior to the administration of the [¹⁸F]-FACBC to a subject, step (a) in each and every aspect of the invention, the subject may consume no food or calorie-containing drink, for example, consume no food or calorie-containing drink for a period of 4 hours, such as 6, hours, e.g. continuous hours.

The [¹⁸F]-FACBC may be administered as an intravenous bolus injection.

The [¹⁸F]-FACBC may be administered to a subject in an effective detectable amount (e.g. administering an amount of [¹⁸F]-FACBC to the subject that is detectable by PET imaging). An exemplary detectable amount of [¹⁸F]-FACBC is 185±20% MBq, according to some aspects herein.

Potential lesion [¹⁸F]-FACBC uptake (that is, uptake of [¹⁸F]-FACBC by the potential lesion) which is higher than [¹⁸F]-FACBC uptake in reference structures may be indicative of the presence of brain lesions, e.g., indicative of the potential lesion being a brain lesion. For example, the [¹⁸F]-FACBC uptake in the potential lesion may be higher than or equal to the [¹⁸F]-FACBC uptake in the parotid grands, and step d) includes determining that the potential lesion is a brain lesion. The brain lesion may be a cancer lesion, e.g., a metastatic cancer lesion. In some examples, the potential lesion may be suspicious for a recurrence of cancer in the subject. In at least one example, the [¹⁸F]-FACBC uptake in the potential lesion is lower than the [¹⁸F]-FACBC uptake in the parotid glands, and step d) includes determining that the potential lesion is not a brain lesion.

In some examples herein, the one or more PET scan images comprise a first PET scan image and at least one subsequent PET scan image, wherein analysing the one or more PET scan images in step c) comprises comparing intensities of [¹⁸F]-FACBC uptake in the first PET scan image and the at least one subsequent PET scan image to determine whether the intensity of [¹⁸F]-FACBC uptake in the at least one subsequent PET scan image has increased, remained constant, or decreased relative to the intensity of [¹⁸F]-FACBC uptake of the first PET scan image.

In some examples herein, the one or more PET scan images comprises a PET scan image at a first location and at least one PET scan image at a subsequent location, and wherein analysing the one or more PET scan images in step c) comprises comparing intensities of [¹⁸F]-FACBC uptake in the PET scan image at the first location and of [¹⁸F]-FACBC uptake in the at least one PET scan image at the subsequent location to determine whether the intensity of the [¹⁸F]-FACBC uptake in the at least one PET scan image at the subsequent location has increased, remained constant, or decreased relative to the intensity of the [¹⁸F]-FACBC uptake in the PET scan image at the first location.

An increase in the relative intensity of [¹⁸F]-FACBC uptake in said at least one subsequent PET scan image relative to the intensity of [¹⁸F]-FACBC uptake of the first PET scan image for a potential lesion, such as a lesion identifiable as a brain cancer lesion according to any aspect of the invention, may be indicative of recurrent brain cancer, such as recurrent metastatic brain cancer.

For example, [¹⁸F]-FACBC uptake in the potential lesion which is greater than the [¹⁸F]-FACBC uptake in the parotid glands and/or pituitary glands may be indicative of the presence of the brain lesion, e.g., indicative of the potential lesion being a brain lesion.

In some embodiments, the potential lesion is a brain lesion that contains diseased tissue and may be a cancer lesion, for example a metastatic cancer lesion.

For diagnosing a brain lesion the reference threshold maximum standardized uptake value (SUV)_{threshold-max} of the [¹⁸F]-FACBC uptake in the potential lesion may be as defined herein. In some embodiments, diagnosis of the absence of a brain lesion, for example a metastatic cancer lesion, may be performed where the maximum standardized uptake value (SUV)ₘₐₓ of the [¹⁸F]-FACBC uptake in the potential lesion is less than said reference threshold maximum standard uptake value (SUV)_{threshold-max}, as defined herein.

For diagnosing a brain lesion the reference threshold peak standardized uptake value (SUV)_{threshold-peak} of the [18F]-FACBC uptake in the potential lesion may be as defined herein. In some embodiments, diagnosis of the absence of a brain lesion, for example a metastatic cancer lesion, may be performed where the peak standardized uptake value (SUV)ₚₑₐₖ of the [18F]-FACBC uptake in the potential lesion is less than said reference threshold maximum standard uptake value (SUV)_{threshold-peak}, as defined herein.

For diagnosing a brain lesion the reference threshold mean standardized uptake value (SUV)_{threshold-mean} of the [18F]-FACBC uptake in the potential lesion may be as defined herein. In some embodiments, diagnosis of the absence of a brain lesion, for example a metastatic cancer lesion, may be performed where the mean standardized uptake value (SUV)ₚₑₐₖ of the [18F]-FACBC uptake in the potential lesion is less than said reference threshold maximum standard uptake value (SUV)_{threshold-mean}, as defined herein.

Diagnosis of a brain lesion, for example a metastatic cancer lesion, may be performed where the potential lesion [¹⁸F]-FACBC uptake (that is, uptake of [¹⁸F]-FACBC by the potential lesion) is equal to or higher than parotid glands [¹⁸F]-FACBC uptake.

In some embodiments of the present disclosure, the potential lesion [¹⁸F]-FACBC uptake is visually compared to [¹⁸F]-FACBC activity in the parotid glands and/or pituitary glands to analyse and determine whether the potential lesion is a metastatic brain lesion in order to diagnose brain metastasis.

According to a further aspect of the invention, there is provided a kit for imaging, diagnosing or monitoring metastatic cancer, comprising: a) [¹⁸F]-FACBC tracer; and b) administration instructions according to any of the aspects described above and/or elsewhere herein.

According to a further aspect of the invention, there is provided the acquisition of images using a PET/MRI or PET/CT scanner. The simultaneous or consecutive acquisition of images on a PET/MRI or PET/CT scanner may offer improved diagnostic accuracy over the generation of images on separate PET and MRI instruments or on separate PET and CT instruments. The conjoint use with MRI may improve the localisation of lesions, particularly where the lesions are small, for example in the case of metastatic lesions. The method of administration can be the methods described herein.

### EXAMPLES

The PET scans in the Examples below were carried out on a variety of PET/CT and PET/MR scanners across the US. Standalone PET scanners were excluded. A standard dose of 5 mCi was injected with a tolerance of ±20%, meaning that the actual dose was between 4 and 6 mCi.

The scanner started acquiring data 30 seconds before the injection of the FACBC and continued to acquire dynamic images for 30 minutes after injection. The frame times were as detailed below—the dynamic acquisition was used so that the images could be rebinned at the various timepoints.

1 frame @ 30 sec interval
12 frames @ 5 sec intervals
6 frames @ 10 sec intervals
6 frames @ 30 sec intervals
5 frames @ 60 sec intervals
4 frames @ 300 sec intervals

For each of the 23 images the reference lesion was sampled surgically and histopathology was carried out. Where ≥10% of the lesion was categorized as recurrent tumour the lesion was categorized as positive for recurrence. By comparing the histopathology to the visual or semi-quantitative readout it was possible to categorise each read as either true positive, true negative, false positive or false negative. Sensitivity and specificity were then calculated using the standard equations (https://www.ncbi.nlm.nih.gov/books/NBK557491/).

### Example 1 (Visual Interpretation)

In this Example, the parotid gland or pituitary as reference organs for visual image interpretation of FACBC images of lesions. Specific instruction is given as to whether to use the parotid gland or pituitary as the reference organ, based on a minimum of 1.0 cm of craniocaudally visualised parotid gland on the scan field of view. If this criterion is met, the parotid gland is used as the reference organ. If it is not met, the pituitary should be used as the reference organ.

The method of this example instructs the reader to interpret the lesion as recurrence (positive) if the lesion FACBC uptake is equal to or higher than the reference organ, and treatment-related change (negative) if the lesion FACBC uptake is less than the reference organ. Comparison of lesion uptake is specifically intended to be the area of highest FACBC uptake within the lesion, compared to the visual average of the reference organ. Instruction is given to making this comparison and is done based on adjustment of the image contrast (‘windowing’) to a specific visual rendering of the reference organ. Once this is achieved, the visual average of the reference organ is considered to approximately be at the ⅘ point of the image scale (‘lookup table’/‘colour scale’). If the lesion uptake (i.e. the area of highest FACBC uptake within the lesion) is visually equal to or higher than this point, then the lesion meets the criterion for positive.

Using this approach on FACBC brain images obtained at 15-25 minutes post-injection of FACBC, diagnostic performance across majority of 3 readers was found to be high, as demonstrated in Table 1 below:

TABLE 1
Performance of visual reads at the 15-25 minute timepoint.

| 1525 data | R1 | R2 | R3 |
|---|---|---|---|
| Sensitivity (%) | 90 | 90 | 60 |
| Specificity (%) | 77 | 85 | 100 |

### Example 2 (Semi-Quantitative Interpretation)

This method uses the pituitary as reference organ for semi-quantitative image interpretation of FACBC images of lesions.

One volume of interest (VOI) each is drawn around the lesion and a separate VOI is drawn around the reference organ using an image workstation. The level of FACBC uptake within these structures are characterised by the hottest (highest level of FACBC uptake) cubic centimetre within the VOL. The level of uptake is characterised numerically by the standardized uptake value, and can be measured as the peak standardized uptake value (SUVpeak), mean standardized uptake value (SUVmean) or maximum standardized uptake value (SUVmax).

By the new method, the lesion uptake is compared to pituitary uptake, generating an SUV ratio (SUVR_{lesion:pituitary}). This ratio can be calculated using SUVpeak, SUVmean and SUVmax and in combinations with SUVmean or SUVpeak as the denominator i.e. SUVpeak/SUVmean. In this example, SUVpeak of the lesion is calculated as a ratio to SUVpeak of the pituitary, and where the ratio is ≥1, the lesion is classified as positive. Where the ratio is <1, the lesion is classified as negative.

Using this approach on FACBC brain images are obtained at 5-15, 10-20, 15-25, 15-30, and 20-30 minutes post-injection of FACBC. Diagnostic performance is obtained as described in Table 2, with clinically acceptable performance observed at all of these timepoints. (see Table 2 below).

TABLE 2
Performance of semi-quantitative method at different timepoints.

| Timepoint | Sensitivity (%) | Specificity (%) |
|---|---|---|
| 515 | 70 | 85 |
| 1020 | 80 | 85 |
| 1525 | 80 | 85 |
| 1530 | 80 | 85 |
| 2030 | 80 | 69 |

The inherent commercial and medical benefits of this approach are its applicability across a wide range of FACBC uptake times (i.e. the amount of time that can lapse between FACBC injection, and performing the patient's brain scan).

### Example 3 (Composite Interpretation)

This new method incorporates use of the two aforementioned methods, where a reader may elect to use the parotid as reference organ for visual image interpretation, and where there is insufficient parotid gland in the scan field of view, use the SUVR method (which uses the pituitary gland).

Using this approach on FACBC brain images obtained at 10-20 minutes, 15-25 minutes and 20-30 minutes post-injection of FACBC, diagnostic performance across 3 readers was as shown in the tables below. The initial number is when the SUVR result is used instead of the visual read using the pituitary. The number in brackets is the visual read. It should be noted that the 1020 read was carried out by different readers to the later timepoints.

Using this approach on FACBC brain images obtained at 10-20 minutes, 15-25 minutes and 20-30 minutes post-injection of FACBC, diagnostic performance across 3 readers was as shown in the tables below. The initial number is when the SUVR result is used instead of the visual read using the pituitary. The number in brackets is the visual read.

TABLE 3
Performance of reads at the 10-20 minute timepoint. The initial
number is when the SUVR result is used instead of the visual read
using the pituitary. The number in brackets is the visual read.

| 1020 data | R1 | R2 | R3 |
|---|---|---|---|
| Sensitivity (%) | 70 (50) | 70 (50) | 80 (80) |
| Specificity (%) | 100 (100) | 100 (100) | 92 (92) |

TABLE 4
Performance of reads at the 15-25 minute timepoint. The initial
number is when the SUVR result is used instead of the visual read
using the pituitary. The number in brackets is the visual read.

| 1525 data | R1 | R2 | R3 |
|---|---|---|---|
| Sensitivity (%) | 80 (90) | 90 (90) | 80 (60) |
| Specificity (%) | 85 (77) | 85 (85) | 92 (100) |

TABLE 5
Performance of reads at the 20-30 minute timepoint. The initial
number is when the SUVR result is used instead of the visual read
using the pituitary. The number in brackets is the visual read.

| 2030 data | R1 | R2 | R3 |
|---|---|---|---|
| Sensitivity (%) | 80 (80) | 80 (90) | 70 (70) |
| Specificity (%) | 69 (62) | 77 (69) | 85 (92) |

### Example 4 (Alternative Composite Interpretation)

The criteria below have been developed for reference organ selection and PET interpretation for using the composite of visual image interpretation and the SUVR method:
A lesion should be reported as being either positive or negative for recurrent metastasis.
Using the parotid glands as the comparison reference organ, a lesion should be reported as being positive for recurrent metastasis if FACBC uptake is visually equal to or above the parotid glands and reported negative if FACBC uptake is visually lower than the parotid glands.
In cases where both of the parotid glands have been inadequately visualized, the pituitary should be used as the reference organ (i.e., a lesion should be reported as positive if it has FACBC uptake visually equal to or above the pituitary, and negative if FACBC uptake is visually lower than the pituitary).
If the lesion FACBC uptake is visually close to the reference organ, a semi-quantitative ratio (SUVR) should be used for interpretation. This is based on a ratio of the hottest cubic centimeter of FACBC uptake (peak standardized uptake value) within the lesion compared to the pituitary.
If the SUVR is equal to or above 1, the lesion should be reported as being positive for recurrent metastasis.
If the SUVR is less than 1, the lesion should be reported as being negative.

### Example 5

Interpretation Criteria below have been developed for the assessment of parotid organ for the visual interpretation and the composite interpretation:
In general, the superior half of the parotid glands will be in the field of view
If coverage of the parotid glands are more extensive than this, base the assessment on the superior half.
Transaxial PET slice width varies from one scanner to another
Scroll through PET transaxial slices to determine if the parotid is sufficiently visualized. Count the number of slices through the parotid gland omitting the noisy inferior slices at the edge of field of view
Use the scanner slice width to determine if ≥1 cm of parotid is visualized.
The parotid gland Must be FACBC avid (i.e. above surrounding background fat; discernible outline; similar to surrounding muscle uptake is acceptable)
Sufficient visualization of the parotid is to be done on the PET, not on the accompanying structural images (CT/MRI).
After counting slices, work out the craniocaudal extent of parotid coverage using the transaxial slice width
Where both parotid glands are adequately visualized but there is asymmetric glandular FACBC uptake, the more avid gland should be used as the reference.
Where there is asymmetric parotid coverage (one adequately visualized while the contralateral gland is inadequately visualized), the adequately imaged parotid should be used as the reference

### Example 6

Criteria below have been developed for the Windowing step interpretation process:

### Optimal Windowing of PET Images:

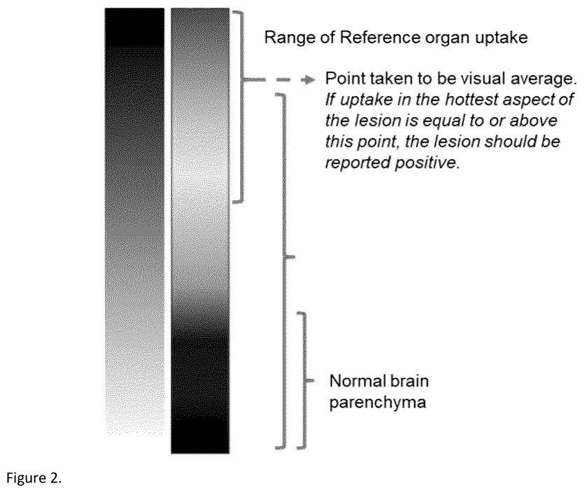

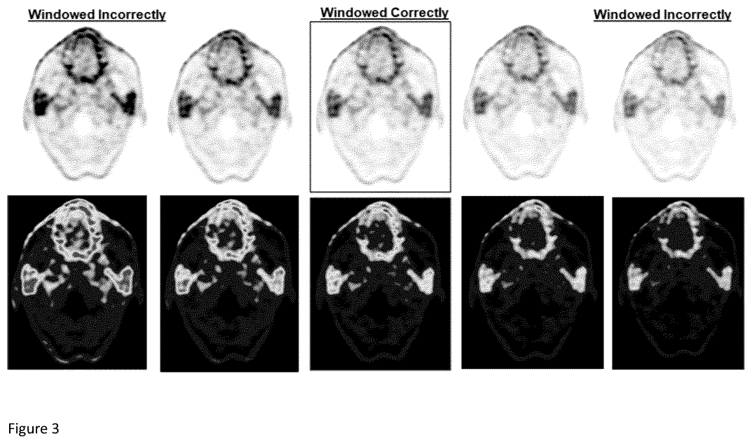

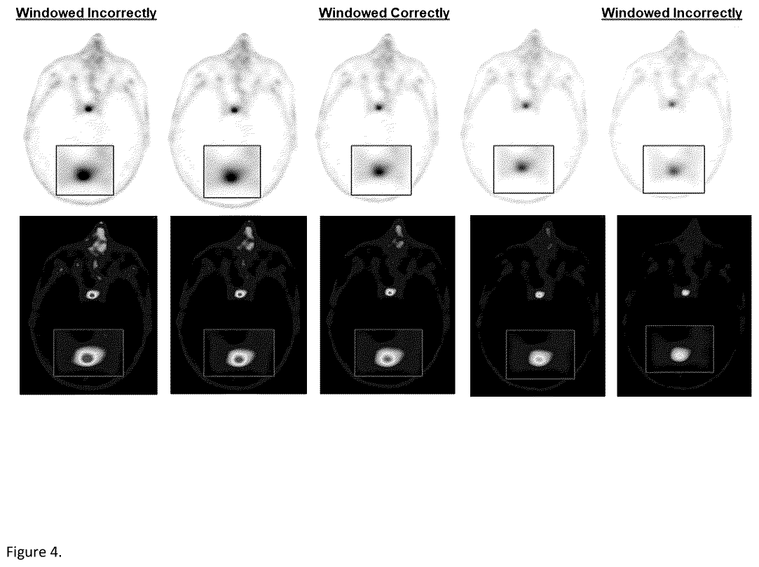

PET images are commonly viewed on an SUV scale. Adjustment of the SUV scale is also referred to as “windowing”.
Optimal windowing avoids over and under interpretation
Use of grayscale, a color scale, or a combination of both, is per reader preference
On initial display of the PET images, the intensity of the reference organ (parotid glands) will likely be saturated. The image will require windowing to lower the intensity of the reference organ prior to the lesion assessment.
The lower SUV threshold will default to zero. Areas of low physiological uptake/activity (normal brain parenchyma and blood pool) will appear on the lower third of the scale.
The upper SUV threshold should be windowed until the hottest areas of the gland are just at the top of the scale, while maintaining tissue definition. Refer to the uptake range of the reference organ (moderate physiological uptake) on the scale in FIG. 2.
Upper Threshold adjusted to the reference organ (parotid gland). As the parotid gland often has heterogeneous uptake, window until the most avid areas of the gland are just at the top of the scale.
Refer to the superior half of the parotid glands, where gland coverage is more extensive
If parotid uptake is asymmetric, window to the gland with higher uptake
Where there is asymmetric parotid coverage, the adequately imaged parotid should be used as the reference (regardless of any difference in uptake to the contralateral gland).
If insufficient parotid in FOV, use the pituitary as the reference organ.
Upper Threshold adjusted to the reference organ (pituitary). Window until the most avid areas of the gland are just at the top of the scale.
Due to the size of the pituitary, magnify the pituitary and use all planes to aid windowing.

1. A method of using [¹⁸F]-FACBC, comprising the steps of:
(a) administering a detectable amount of [18F]-FACBC to a subject;
(b) acquiring one or more positron emission tomography (PET) scan images of a head region of the subject;
(c) visually analysing the one or more PET scan images by comparing [18F]-FACBC uptake in a potential lesion of the head region of the subject against [18F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [18F]-FACBC uptake in a potential lesion of the head region of the subject against [18F]-FACBC uptake of the parotid gland. If criterion of (i) is not met, comparing [18F]-FACBC uptake in a potential lesion of the head region of the subject against [18F]-FACBC uptake of the pituitary in the head region, and
(d) determining whether the potential lesion is a brain lesion based on the analysis of step (c).

2. The method of claim 1, wherein the brain lesion is a metastatic brain lesion.

3. The method of claim 1, wherein the predetermined threshold of the craniocaudally visualised parotid gland on the scan field of view is between 0.8-1.2 cm.

4. The method of claim 3, where in the predetermined threshold of the craniocaudally visualised parotid gland on the scan field of view is about 1 cm.

5. The method of claim 1, wherein FACBC brain images of the methods are obtained about 15-25 minutes post-injection of FACBC.

6. A method of using [¹⁸F]-FACBC, comprising the steps of:
(a) administering a detectable amount of [¹⁸F]-FACBC to a subject;
(b) acquiring one or more positron emission tomography (PET) scan images of a head region of the subject;
(c) analysing the one or more PET scan images by comparing a standardized uptake value (SUV) of [¹⁸F]-FACBC uptake in a potential lesion against a reference threshold standardized uptake value (SUV)_{threshold} of the pituitary glands; and
(d) determining whether the potential lesion is a brain lesion based on the analysis of step (c).

7. The method of claim 6, wherein the brain lesion is a metastatic brain lesion.

8. The method of any claims of claim 6, wherein FACBC brain images of the methods are obtained about 15-25 minutes post-injection of FACBC.

9. A method of using [¹⁸F]-FACBC, comprising the steps of:
(a) administering a detectable amount of [18F]-FACBC to a subject;
(b) acquiring one or more positron emission tomography (PET) scan images of a head region of the subject;
(c) visually analysing the one or more PET scan images by comparing [18F]-FACBC uptake in a potential lesion of the head region of the subject against [18F]-FACBC uptake in the parotid glands or pituitary of the head region by (i) determining whether the criterion of a minimum of predetermined size of craniocaudally visualised parotid gland on the scan field of view is met; and (ii), if the criterion of (i) is met, comparing [18F]-FACBC uptake in a potential lesion of the head region of the subject against [18F]-FACBC uptake of the parotid gland. If criterion of (i) is not met, comparing [18F]-FACBC uptake in a potential lesion of the head region of the subject against [18F]-FACBC uptake of the pituitary in the head region;
(d) if in step (c) the [18F]-FACBC uptake in a potential lesion of the head region of the subject is visually close to [18F]-FACBC uptake in parotid glands or pituitary of the head region, comparing a standardized uptake value (SUV) of [18F]-FACBC uptake in a potential lesion against a reference threshold standardized uptake value (SUV)threshold of the pituitary, and
(e) determining whether the potential lesion is a brain lesion based on the analysis of step (c) and (d).

10. The method of claim 9, wherein the brain lesion is a metastatic brain lesion.

11. The method of claim 9, wherein the predetermined threshold of the craniocaudally visualised parotid gland on the scan field of view is between 0.8-1.2 cm.

12. The method of claim 9, where in the predetermined threshold of the craniocaudally visualised parotid gland on the scan field of view is about 1 cm.

13. The method of claim 9, wherein FACBC brain images of the methods are obtained about 15-25 minutes post-injection of FACBC.
